(19) United States
(12) Patent Application Publication
(10) Pub. No.: US 2022/0151649 A1
(43) Pub. Date: May 19, 2022

(54) SURGICAL FORCEPS

(71) Applicant: THE UNIVERSITY OF TOKYO, Tokyo (JP)

(72) Inventor: Koji IKUTA, Tokyo (JP)

(73) Assignee: THE UNIVERSITY OF TOKYO, Tokyo (JP)

(21) Appl. No.: 17/425,975

(22) PCT Filed: Jan. 24, 2020
(86) PCT No.: PCT/JP2020/002444

§ 371 Date: Jul. 27, 2021

(30) Foreign Application Priority Data
Jan. 31, 2019 (JP) 2019-015861

(51) Int. Cl. A61B 17/29

(52) U.S. Cl. CPC A61B 17/2909; A61B 2017/00867

(57) ABSTRACT

A working portion, a support portion, a bending portion, a linear portion and an operating portion connected with one another in this sequence. The operating portion includes a working operation portion configured to operate the working portion and a bending operation portion configured to operate bending of the bending portion. A single rod-like super-elastic wire that is made of a metal material having super-elasticity and that has a diameter of not larger than 0.5 mm is used as a working wire having one end mounted to the working portion and the other end mounted to the working operation portion. The super-elastic wires are also used as there or more bending wires having respective one ends mounted to the support portion and respective other ends mounted to the bending operation portion.

### TECHNICAL FIELD

The present disclosure relates to a surgical forceps.

### BACKGROUND

A conventionally proposed technique is a medical manipulator having a working portion configured to perform work such as gripping, a first bending portion, and a second bending portion (as shown in, for example, Patent Literature 1). In this medical manipulator, a guide ring in a neighborhood of the working portion and an operating portion are connected with each other by a plurality of bending wires that are arranged at equal intervals on a concentric circle, and the first bending portion is bent by pulling one or multiple bending wires.

### CITATION LIST

### Patent Literature

PTL1: JP2014-265A

### SUMMARY

### Technical Problem

The surgical forceps are desired to be as thin as possible as a whole, as well as to be readily bent in any arbitrary direction with respect to two directions in the vicinity of the working portion, with a view to allowing the working portion that performs work such as gripping to reach a location where the work is required. A wire is generally enabled to apply a desired axial force with regard to a tensile force but is not enabled to apply a desired axial force with regard to a compressive force, due to buckling. Accordingly, two wires are required for the work of the working portion, and three or more wires are required for bending.

A main object of the present disclosure is to provide surgical forceps that are readily bendable in any arbitrary direction with respect to two directions in a neighborhood of a working portion and that are thin as a whole.

### Solution to Problem

In order to achieve the above main object, the surgical forceps of the disclosure is implemented by an aspect described below.

The present disclosure is directed to a surgical forceps including a working portion, a support portion configured to support the working portion, a bending portion configured to be bendable, a linear portion configured not to be bended, and an operating portion, which are connected with one another in this sequence. The operating portion includes a working operation portion configured to operate the working portion and a bending operation portion configured to operate bending of the bending portion. The surgical forceps further includes a working wire formed by a single rod-like super-elastic wire that is made of a metal material having super-elasticity and that has a diameter of not larger than 0.5 mm, configured to have one end mounted to the working portion and the other end mounted to the working operation portion, and placed inside of the support portion, the bending portion, and the linear portion to be freely movable in an axial direction, and bending wires formed by three or more rod-like super-elastic wires that are made of a metal material having super-elasticity and that have diameters of not larger than 0.5 mm, configured to have respective one ends mounted to the support portion and respective other ends mounted to the bending operation portion, and placed inside of the bending portion and the linear portion to be freely movable in an axial direction.

In the surgical forceps of this aspect, the working portion, the support portion configured to support the working portion, the bending portion configured to be bendable, the linear portion configured not to be bended, and the operating portion including the working operation portion configured to operate the working portion and the bending operation portion configured to operate bending of the bending portion are connected with one another in this sequence. The working wire configured to have one end mounted to the working portion and the other end mounted to the working operation portion and placed inside of the support portion, the bending portion, and the linear portion to be freely movable in the axial direction is formed by the single rod-like super-elastic wire that is made of the metal material having super-elasticity and that has the diameter of not larger than 0.5 mm. The bending wires configured to have respective one ends mounted to the support portion and respective other ends mounted to the bending operation portion and placed inside of the bending portion and the linear portion to be freely movable in the axial direction are formed by three or more rod-like super-elastic wires that are made of the metal material having super-elasticity and that have the diameters of not larger than 0.5 mm. The super-elastic wire is formed in a rod-like shape and is thus enabled to apply a compressive force-based axial force, as well as a tensile force-based axial force. This configuration enables a tensile force-based behavior and a compressive force-based behavior to be performed by one single wire as behaviors in the operating portion. This decreases the number of wires used in the surgical forceps and reduces the thickness of the forceps. The super-elastic wire has a wide area of elastic deformation and accordingly allows for a large bent and a return thereof. This configuration enables the surgical forceps to be readily bent in any arbitrary direction with respect to two directions in the bending portion in the vicinity of the working portion. As a result, the surgical forceps of this aspect are readily bendable in any arbitrary direction with respect to the two directions in the vicinity of the working portion and are additionally thin as a whole. The super-elastic wire is required to be formed in a rod-like shape having the diameter of not larger than 0.5 mm from the metal material. The super-elastic wire having the diameter of not larger than 0.3 mm or having the diameter of not larger than 0.2 mm is also preferable.

An example of the metal material having super-elasticity is an alloy of titanium (Ti) and nickel (Ni). In the operating portion, the working operation portion may be formed as a working member, and the bending operation portion may be formed as a bending member. The working member may have a behavior in one direction and have a behavior in an opposite direction to apply a tensile force and a compressive force to the working wire, so as to drive the working portion. The bending member may have an inclination in any arbitrary direction with respect to two directions perpendicular to the axial direction of the wire to apply a compressive force to the wire on the inner side and apply a tensile force to the wire on the outer side, so as to bend the bending portion. The operating portion may be configured by an electric actuator configured to individually apply a tensile force and a compressive force to each of the wires.

In the surgical forceps of the above aspect, the bending wires may be configured by a number of wires corresponding to a number of apexes of a regular polygon, which are arranged to form the respective apexes of the regular polygon. In this aspect, when the bending operation portion is configured by a single member, inclining the bending operation portion in any arbitrary direction with respect to two directions perpendicular to the axial direction of the applies a compressive force to the wire on an inner side and applies a tensile force to the wire on an outer side, so as to freely bend the bending portion.

In the surgical forceps wherein the bending wires are arranged to form the respective apexes of the regular polygon of the above aspect, the bending wires may be arranged not to be twisted in the linear portion. When the bending operation portion is configured by a single member, the configuration of this aspect enables the bending portion to be bent in an opposite direction to an inclination direction of the bending operation portion. Further, the bending wires may be twisted by 180 degrees in the linear portion. When the bending operation portion is configured by a single member, the configuration of this aspect enables the bending portion to be bent in an identical direction with an inclination direction of the bending operation portion.

In the surgical forceps wherein the bending wires are arranged to form the respective apexes of the regular polygon of the above aspect, the working wire may be arranged to form a center of the regular polygon. The configuration of this aspect enables the working wire to be placed in a vacant space of the bending wires and thereby provides thin forceps.

In the surgical forceps of the above aspect, the working portion may include a stationary portion and a movable portion that is mounted to the stationary portion to be freely rotatable by means of a hinge, and the working wire may be mounted and fixed at a position eccentric from the hinge of the movable portion. The configuration of this aspect enables a rotational motion of the movable portion in one direction and a rotational motion of the movable portion in an opposite direction to be performed by applying a tensile force-based axial force and a compressive force-based axial force to the working wire.

### BRIEF DESCRIPTION OF DRAWINGS

FIG. 1 is an explanatory diagram schematically illustrating the configuration of surgical forceps 20 according to one embodiment of the present disclosure;

FIG. 2 is an enlarged schematic diagram schematically illustrating closeup of the working portion 30, support portion 40 and bending portion 50;

FIG. 3 is a sectional view illustrating one example of a section of a guide member 62 taken along a line A-A in FIG. 2;

FIG. 4 is a sectional view illustrating one example of a section of the support member 40 taken along a line B-B in FIG. 2;

FIG. 5 is an explanatory diagram illustrating the state that the bending portion 50 is bent in the sections of the bending wires 94a and 94c;

FIG. 6 is an explanatory diagram illustrating the state that a movable member 34 of the working portion 30 is being moved;

FIG. 7 is an explanatory diagram schematically illustrating the configuration of the working operation portion 74;

FIG. 8 is an explanatory diagram schematically illustrating closeup of the operation base member 72 and the bending operation portion 80 in the cross section of the bending wires 94a and 94c;

FIG. 9 is an explanatory diagram illustrating one example of a cross section of the guide member 84;

FIG. 10 is an explanatory diagram schematically illustrating closeup of the operation base member 72 and the bending operation portion 80 at the cross sections of the bending wires 94a and 94c in the state that the operation knob 82 is operated, and

FIG. 11 is an explanatory diagram schematically illustrating the configuration of surgical forceps 120 according to a modification.

### DESCRIPTION OF EMBODIMENTS

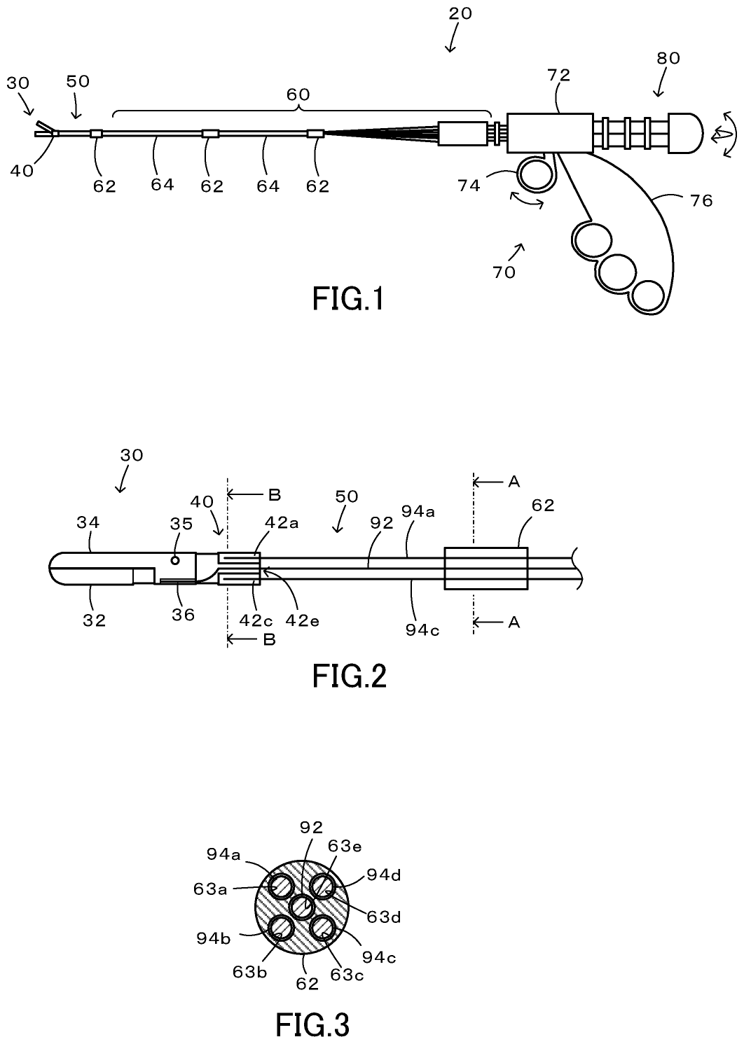

The following describes some embodiments of the present disclosure. FIG. 1 is an explanatory diagram schematically illustrating the configuration of surgical forceps 20 according to an embodiment. The surgical forceps 20 of the embodiment include, from the left side in the drawing, a working portion 30 configured to perform work such as gripping; a support portion 40 mounted to the working portion 30; a bending portion 50 configured to be bendable in any arbitrary direction with respect to two directions that are a vertical direction and a front-rear direction in the drawing; a linear portion 60; and an operating portion 70.

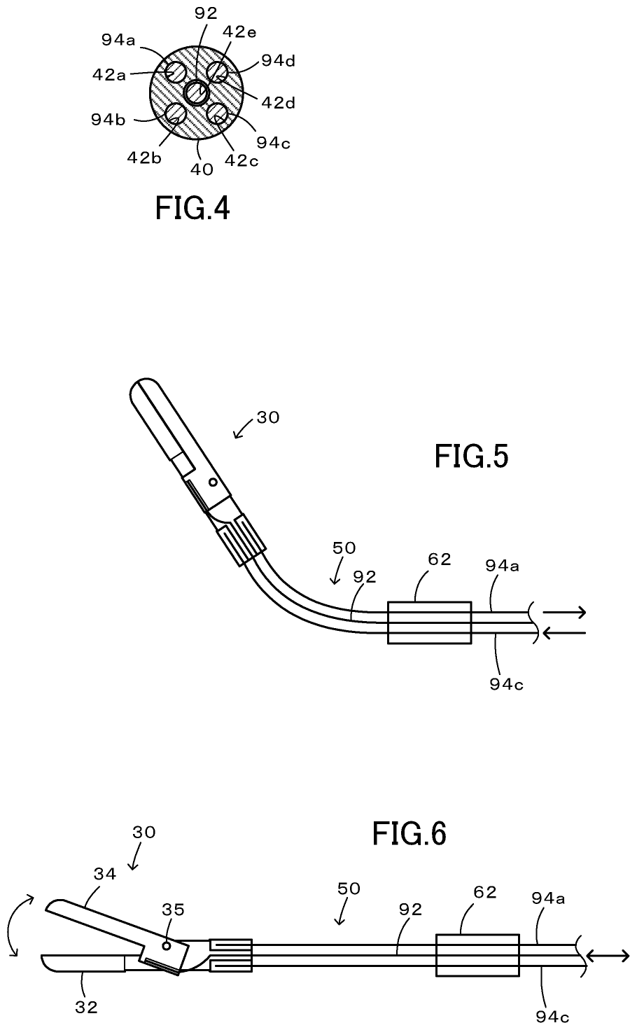

FIG. 2 is an enlarged schematic diagram schematically illustrating closeup from the working portion 30 to part of the linear portion 60. FIG. 3 is a sectional view illustrating one example of a section of a guide member 62 taken along a line A-A in FIG. 2. FIG. 4 is a sectional view illustrating one example of a section of the support portion 40 taken along a line B-B in FIG. 2.

The linear portion 60 includes a plurality of guide members 62 and hollow pipe members 64 provided to connect these guide members 62 with each other. As shown in FIG. 3, the guide member 62 includes four through holes 63a to 63d formed at equal intervals on a concentric circle (to form respective apexes of a square) and one through hole 63e formed at the center of these four through holes 63a to 63d. The four through holes 63a to 63d are formed to have inner diameters that are slightly larger than the diameters of respective bending wires 94a to 94d. The four bending wires 94a to 94d provided to bend the bending portion 50 are arranged to be guided movably in an axial direction relative to the through holes 63a to 63d. The through hole 63e at the center is formed to have an inner diameter that is slightly larger than the diameter of a working wire 92. The working wire 92 provided to drive the working portion 30 is arranged to be guided movably in an axial direction relative to the through hole 63e. The plurality of guide members 62 are arranged to align the respective four through holes 63a to 63d, so that the four bending wires 94a to 94d connect the bending portion 50 with the operating portion 70 without being twisted. Each of the working wire 92 and the four bending wires 94a to 94d is formed as a single rod-like super-elastic wire that is made of a metal material having super-elasticity (for example, an alloy of titanium (Ti) and nickel (Ni)) and that has a diameter of not larger than 0.2 mm. Each of the working wire 92 and the four bending wires 94a to 94d is formed in a rod-like shape from the metal material having super-elasticity and is thus enabled to apply a compressive force-based axial force, as well as a tensile force-based axial force. Each of the working wire 92 and the four bending wires 94a to 94d has super-elasticity and thereby has a wide area of elastic deformation to allow for a large bend and a return thereof. The guide member 62 is required to have the four through holes 63a to 63d formed on a concentric circle and the through hole 63e formed at the center of the four holes 63a to 63d, such as to cause the four bending wires 94a to 94d having the diameter of not larger than 0.2 mm and the working wire 92 having the diameter of not larger than 0.2 mm to be movable in the axial direction. The guide member 62 is thus formed as a member having a diameter of not larger than 2 mm (having a diameter of approximately 1 mm according to the embodiment). Accordingly, the hollow pipe member 64 is also formed as a member having a diameter of not larger than 2 mm (having a diameter of approximately 1 mm according to the embodiment).

The support portion 40 is integrally formed with a stationary member 32 of the working portion 30 described later and includes four non-through holes 42a to 42d formed at equal intervals on a concentric circle (to form respective apexes of a square) and a through hole 42e formed at the center of these four holes 42a to 42d, as shown in FIG. 4. The four holes 42a to 42d are formed to have inner diameters that are slightly smaller than the diameters of the four bending wires 94a to 94d. The four bending wires 94a to 94d are mounted and fixed by press fitting respective ends of the four bending wires 94a to 94d into the four holes 42a to 42d. The through hole 42e at the center is formed to have an inner diameter that is slightly larger than the diameter of the working wire 92 and is arranged such that the working wire 92 is guided to be movable in the axial direction relative to the through hole 42e. The support portion 40 is formed to have a diameter of not larger than 2 mm (to have a diameter of approximately 1 mm according to the embodiment), like the guide member 62.

The working portion 30 includes a stationary member 32 that is formed integrally with the support portion 40 to be not movable; and a movable member 34 mounted to the stationary member 32 to be freely rotatable by means of a hinge 35. The movable member 34 has a hole 36 formed at a position eccentric from a hinge 35 (at a lower position in FIG. 2). The working wire 92 is mounted and fixed by press fitting an end of the working wire 92 into this hole 36. The working portion 30 of the embodiment is formed to have a diameter of not larger than 2 mm in the state that the movable member 34 is aligned with the stationary member 32.

FIG. 5 is an explanatory diagram illustrating the state that the bending portion 50 is bent in the sections of the bending wires 94a and 94c. Arrows at a right end in FIG. 5 indicate moving directions of the bending wires 94a and 94c. FIG. 2 schematically illustrates closeup from the working portion 30 to part of the linear portion 60 in the sections of the bending wires 94a and 94c. Applying a tensile force from the guide member 62-side to the bending wire 94a and a compressive force from the guide member 62-side to the bending wire 94c in the state of FIG. 2 causes the bending wire 94a to be guided by the guide member 62 and to be moved rightward in the drawing, while causing the bending wire 94c to be guided by the guide member 62 and to be moved leftward in the drawing. The bending portion 50 is accordingly bent toward the bending wire 94a-side as shown in FIG. 5. Applying a compressive force to the bending wire 94a and tensile force to the bending wire 94c in this state (in the state of FIG. 5) causes the bending wire 94a to be guided by the guide member 62 and to be moved leftward in the drawing, while causing the bending wire 94c to be guided by the guide member 62 and to be moved rightward in the drawing. This returns the bending portion 50 to the state of FIG. 2. Further applying a compressive force to the bending wire 94a and a tensile force to the bending wire 94c causes the bending portion 50 to be bent toward the bending wire 94c (downward in FIG. 2). As shown in FIG. 3 and FIG. 4, the bending wires 94b and 94d are arranged at positions different by 90 degrees from the bending wires 94a and 94c. Applying a tensile force to the bending wire 94b and a compressive force to the bending wire 94d from the guide member 62-side causes the bending portion 50 to be bent in the front-back direction in FIG. 2. Accordingly, combination of the bending by the bending wires 94a and 94c with the bending by the bending wires 94b and 94d enables the bending portion 50 to be bent in any arbitrary direction with respect to two directions perpendicular to the linear portion 60. The degree of bending of the bending portion 50 depends on the moving amounts of the bending wires 94a to 94d.

FIG. 6 is an explanatory diagram illustrating the state that the movable member 34 of the working portion 30 is being moved. An arrow at a left end of FIG. 6 indicates a moving direction, and an arrow at a right end indicates moving of the working wire 92. Applying a compressive force from the guide member 62-side to the working wire 92 in the state of FIG. 2 causes the working wire 92 to be guided by the through hole 42e of the support portion 40, to be moved leftward in the drawing, and to press the hole 36 of the movable member 34 leftward in the drawing. The movable member 34 is mounted to the stationary member 32 to be freely rotatable by means of the hinge 35. The movable member 34 is accordingly rotated and driven about the hinge 35 as its rotating axis such as to move one end (a left end in the drawing) of the movable member 34 upward in the drawing to the state of FIG. 6. Applying a tensile force from the guide member 62-side to the working wire 92 in this state (in the state of FIG. 6) causes the working wire 92 to be guided by the through hole 42e of the support portion 40, to be moved rightward in the drawing, and to pull back the hole 36 of the movable member rightward in the drawing. The movable member 34 is accordingly rotated and driven about the hinge 35 as its rotating axis such as to move one end (the left end in the drawing) of the movable member 34 downward in the drawing and to be returned to the state of FIG. 2. A rotational angle (driving amount) of the movable member 34 depends on the moving amount of the working wire 92.

The operating portion 70 includes an operation base member 72, a working operation portion 74 configured to drive the working portion 30, a hand grip 76 mounted and fixed to the operation base member 72, and a bending operation portion 80 configured to bend the bending portion 50.

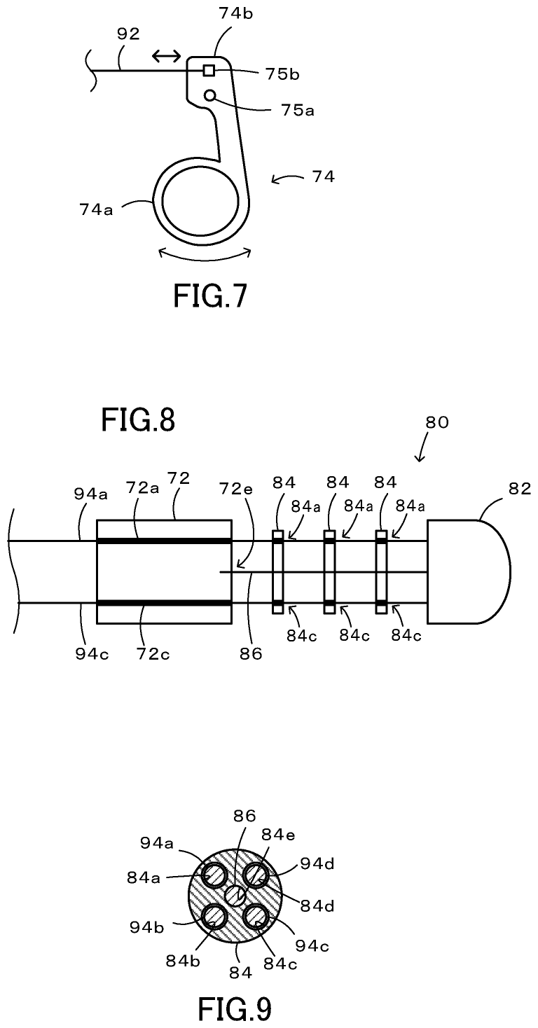

FIG. 7 is an explanatory diagram schematically illustrating the configuration of the working operation portion 74. The working operation portion 74 includes a trigger portion 74a that is protruded downward from the operation base member 72 to be operated; and a cam portion 74b placed in the operation base member 72 and mounted to the operation base member 72 to be rotatable and drivable by means of the hinge 75a. A mounting fixation element 75b provided to mount and fix the working wire 92 is formed at a position eccentric from a hinge 75a of the cam portion 74b (at a position above the hinge 75a in the drawing). One end of the working wire 92 is mounted and fixed to the mounting fixation element 75b. When the trigger portion 74a is moved rightward in the drawing, the working operation portion 74 is rotated anticlockwise about the hinge 75a to press the working wire 92 leftward in the drawing. The working wire 92 is accordingly moved leftward in FIG. 2 and in FIG. 6 by the guide member 62 to change the working portion 30 from the state of FIG. 2 to the state of FIG. 6. When the trigger portion 74a is moved leftward in the drawing, on the other hand, the working operation portion 74 is rotated clockwise about the hinge 75a to pull the working wire 92 rightward in the drawing. The working wire 92 is accordingly moved rightward in FIG. 2 and in FIG. 6 by the guide member 62 to change the working portion 30 from the state of FIG. 6 to the state of FIG. 2.

FIG. 8 is an explanatory diagram schematically illustrating closeup of the operation base member 72 and the bending operation portion 80 in the cross section of the bending wires 94a and 94c. The operation base member 72 includes four through holes 72a to 72d formed at equal intervals on a concentric circle (to form respective apexes of a square). The four through holes 72a to 72d are formed to have inner diameters that are slightly larger than the diameters of the bending wires 94a to 94d and are arranged such as to guide the four bending wires 94a to 94d movably in the axial direction relative to the through holes 72a to 72d. A non-through hole 72e is formed at the center of the four through holes 72a to 72d on a bending operation portion 80-side end of the operation base member 72. A guide wire 86 made of a super-elastic wire has one end press fit into the hole 72e, so as to be mounted and fixed.

The bending operation portion 80 includes a plurality of guide members 84 and an operation knob 82 located on one end thereof. FIG. 9 is an explanatory diagram illustrating one example of a cross section of the guide member 84. As shown in FIG. 9, the guide member 94 includes four through holes 84a to 84d formed at equal intervals on a concentric circle (to form respective apexes of a square) and one through hole 84e formed at the center of these four through holes 84a to 84d. The four through holes 84a to 84d are formed to have inner diameters that are slightly larger than the diameters of the respective bending wires 94a to 94d and are arranged such as to guide the four bending wires 94a to 94d movably in the axial direction relative to the through holes 84a to 84d. The through hole 84e at the center is formed to have an inner diameter that is slightly smaller than the diameter of the guide wire 86. The guide wire 86 is press fit into the through hole 84e, so as to be mounted and fixed. The operation knob 82 includes non-illustrated four non-through holes formed on a concentric circle on one end thereof (on a left end in FIG. 8) and one hole formed at the center of these four holes. The respective ends of the four bending wires 94a to 94d and the end of the guide wire 86 are press fit into these five holes, so that the operation knob 82 is mounted and fixed.

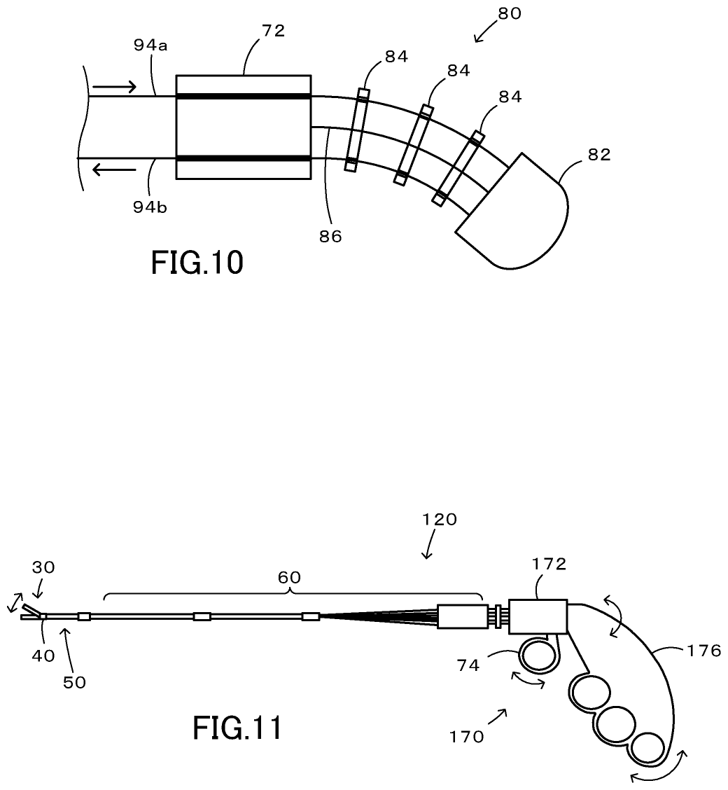

FIG. 10 is an explanatory diagram schematically illustrating closeup of the operation base member 72 and the bending operation portion 80 at the cross sections of the bending wires 94a and 94c in the state that the operation knob 82 is operated. When the operation knob 82 is operated in the state of FIG. 8 toward the bending wire 94c-side (downward in FIG. 8) to the state of FIG. 10, since the guide wire 86 is mounted and fixed to the operation base member 72, the plurality of guide members 84 and the operation knob 82, a tensile force is applied to the bending wire 94a on an outer circumferential side. The bending wire 94a is then guided by the through hole 72a of the operation base member 72 and by the through holes 84a of the plurality of guide members 84 to be moved toward the operation knob 82-side (rightward in FIG. 10). A compressive force is, on the other hand, applied to the bending wire 94c on an inner circumferential side. The bending wire 94c is accordingly guided by the through hole 72c of the operation base member 72 and the through holes 84c of the plurality of guide members 84 to be moved toward the linear portion 60-side (leftward in FIG. 10). This changes the state of the bending portion 50 from the state of FIG. 2 to the state of FIG. 5. When the operation knob 82 is operated in the state of FIG. 10 toward the bending wire 94a-side (upward in FIG. 10), a compressive force is applied to the bending wire 94a, and the bending wire 94a is guided by the through hole 72a of the operation base member 72 and by the through holes 84a of the plurality of guide members 84 to be moved toward the linear portion 60-side (leftward in FIG. 10). A tensile force is applied to the bending wire 94c, and the bending wire 94c is guided by the through hole 72c of the operation base member 72 and by the through holes 84c of the plurality of guide members 84 to be moved toward the operation knob 82-side (rightward in FIG. 10). This changes the state of the bending portion 50 from the state of FIG. 5 to the state of FIG. 2. When the operation knob 82 is further operated toward the bending wire 94a-side (upward in FIG. 10), the bending portion 50 is bent toward the bending wire 94c-side (on the opposite side to FIG. 5). Accordingly, when the operation knob 82 is operated in the vertical direction in FIG. 8, the bending portion 50 is bent in an opposite direction to the operating direction of the operation knob 82. As shown in FIG. 9, the bending wires 94b and 94d are placed at positions different from the bending wires 94a and 94c by 90 degrees. When the operation knob 82 is operated in the front-rear direction in FIG. 8, a tensile force and a compressive force are applied to the bending wires 94b and 94d, and the bending wires 94b and 94d are guided by the through hole 72c of the operation base member 72 and by the through holes 84c of the plurality of guide members 84 to be moved in opposite directions with respect to a left-right direction in FIG. 8. The bending portion 50 is accordingly bent in an opposite direction to the operating direction of the operation knob 82. Combination of bending by the operation of the operation knob 82 in the vertical direction in FIG. 8 with bending by the operation of the operation knob 82 in the front-rear direction in FIG. 8 enables the bending portion 50 to be bent in any arbitrary direction with respect to two directions perpendicular to the linear portion 60. The operation of the operation knob 82 enables the bending portion 50 to be bent in the opposite direction to the operating direction of the operation knob 82. The moving amounts of the bending wires 94a to 94d by the operation of the operation knob 82 depend on the operation amount of the operation knob 82, so that the degree of bending of the bending portion 50 is adjustable by the operation amount of the operation knob 82.

In the surgical forceps 20 of the embodiment described above, the rod-like super-elastic wires that are made of the metal material having the super-elasticity and that have the diameter of not larger than 0.2 mm are used as the working wire 92 and the four bending wires 94a to 94d. This configuration decreases the number of wires required to drive the working portion 30 and thereby reduces the thickness of the forceps. Furthermore, using the super-elastic wires formed in the rod-like shape from the metal material having super-elasticity as the working wire 92 and the four bending wires 94a to 94d enables the working wire 92 and the four bending wires 94a to 94d to apply a compressive force-based axial force as well as a tensile force-based axial force and thereby enables the bending portion 50b to be bent smoothly. As a result, this configuration enables the bending portion 50 to be readily bent in any arbitrary direction with respect to two directions and provides the thin surgical forceps as a whole.

In the surgical forceps 20 of the embodiment, the bending portion 50 is bent in the opposite direction to the operating direction of the operation knob 82 by the operation of the operation knob 82. The bending portion 50 may, however, be bent in an identical direction with the operating direction of the operation knob 82 by the operation of the operation knob 82. In this modification, the four bending wires 94a to 94d as a whole are required to be twisted by 180 degrees in the linear portion 60. More specifically, among the plurality of guide members 62, the phases of all the guide members 62 located on the operating portion 70-side from any arbitrary guide member 62 on the operating portion 70-side relative to the guide member 62 connected with the bending portion 50 are differed from the phase of the guide member 62 connected with the bending portion 50 by 180 degrees.

In the surgical forceps 20 of the embodiment, the bending portion 50 is bent by the four bending wires 94a to 94d. The bending portion 50 may, however, be bent by three bending wires or may be bent by five or more bending wires. In this modification, with respect to the support portion 40, the guide member 62, and the guide member 84, in the case of using three bending wires, the three bending wires should be arranged at equal intervals on a concentric circle (to form apexes of an equilateral triangle), and in the case of using five or more bending wires, the five or more bending wires should be arranged at equal intervals on a concentric circle (to form respective apexes of a regular polygon).

In the surgical forceps 20 of the embodiment, each of the working wire 92 and the four bending wires 94a to 94d is formed as the single rod-like super-elastic wire that is made of the metal material having super-elasticity and has the diameter of not larger than 0.2 mm. The dimeter is, however, not limited to 0.2 mm or smaller but may be 0.3 mm or may be 0.5 mm. Accordingly, the diameter may be not larger than 0.5 mm, and the diameter of not larger than 0.3 mm or the diameter of not larger than 0.2 mm is more preferable.

The surgical forceps 20 of the embodiment are provided with the working operation portion 74, the hand grip 76 and the bending operation portion 80. Like an operating portion 170 of surgical forceps 120 of a modification illustrated in FIG. 11, a hand grip 176 may also work as the bending operation portion. More specifically, the configuration of the bending operation portion 80 other than the operation knob 82 is provided inside of an operation base member 172, and the four bending wires 94a to 94d and the guide wire 86 are mounted and fixed to the hand grip 176 in a similar manner to the mounting to the operation knob 82. In this modification, an operation of the hand grip 176 similar to the operation of the operation knob 82 enables the bending portion 50 to be bent by a degree corresponding to an operation amount in an opposite direction to an operating direction of the hand grip 176 (in the case of twisting the wires in the linear portion 60, in an identical direction with the operating direction).

The surgical forceps 20 of the embodiment are provided with the working operation portion 74, the hand grip 76 and the bending operation portion 80. The surgical forceps 20 may, however, be provided with an electric actuator for working to apply a tensile force and a compressive force to the working wire 92 and with an electric actuator for bending to apply a tensile force and a compressive force to each of the four bending wires 94a to 94d.

In the surgical forceps 20 of the embodiment, the linear portion 60 and the operating portion 70 are directly connected with each other. The linear portion 60 and the operating portion 70 may, however, not be directly connected with each other. In this case, each of the working wire 92 and the four bending wires 94a to 94d may be guided movably in the axial direction in a guide pipe that is not elongated or contracted in the axial direction, such as to allow each wire to be curved with a small clearance.

Some aspects of the present disclosure are described above with reference to the embodiments. The present disclosure is, however, not limited to these embodiments but may be implemented by a variety of other aspects within the scope of the present disclosure.

### INDUSTRIAL APPLICABILITY

The present disclosure is applicable to, for example, manufacturing industry of surgical forceps.

1. Surgical forceps, comprising a working portion, a support portion configured to support the working portion, a bending portion configured to be bendable, a linear portion configured not to be bended, and an operating portion, which are connected with one another in this sequence, wherein
the operating portion includes a working operation portion configured to operate the working portion and a bending operation portion configured to operate bending of the bending portion,
the surgical forceps further comprising:
a working wire formed by a single rod-like super-elastic wire that is made of a metal material having super-elasticity and that has a diameter of not larger than 0.5 mm, configured to have one end mounted to the working portion and the other end mounted to the working operation portion, and placed inside of the support portion, the bending portion, and the linear portion to be freely movable in an axial direction; and
bending wires formed by three or more rod-like super-elastic wires that are made of a metal material having super-elasticity and that have diameters of not larger than 0.5 mm, configured to have respective one ends mounted to the support portion and respective other ends mounted to the bending operation portion, and placed inside of the bending portion and the linear portion to be freely movable in an axial direction.

2. The surgical forceps according to claim 1,
wherein the bending wires are configured by a number of wires corresponding to a number of apexes of a regular polygon, which are arranged to form the respective apexes of the regular polygon.

3. The surgical forceps according to claim 2,
wherein the bending wires are arranged not to be twisted in the linear portion.

4. The surgical forceps according to claim 2,
wherein the bending wires are twisted by 180 degrees in the linear portion.

5. The surgical forceps according to claim 2,
wherein the working wire is arranged to form a center of the regular polygon.

6. The surgical forceps according to claim 1,
wherein the working portion includes a stationary portion and a movable portion that is mounted to the stationary portion to be freely rotatable by means of a hinge, and
the working wire is mounted and fixed at a position eccentric from the hinge of the movable portion.
